(19) United States
(12) Patent Application Publication
(10) Pub. No.: US 2006/0041270 A1
(43) Pub. Date: Feb. 23, 2006

(54) Medical access sheath

(76) Inventors: Jay Lenker, Laguna Beach, CA (US); Onnik Tchulluian, Carlsbad, CA (US); Edward J. Nance, Corona, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/124,903

(22) Filed: May 9, 2005

Related U.S. Application Data
(60) Provisional application No. 60/569,519, filed on May 7, 2004.

(51) Int. Cl. A61M 29/00

(52) U.S. Cl. 606/198

(57) ABSTRACT

An expandable sheath is insertable into a patient through an incision. Once inserted and advanced to the target surgical site, the sheath can be expanded to an enlarged diameter. The wall of the sheath is fabricated from a tubular structure comprising filamentous elements that extend axially and at least partially circumferentially along the length of the sheath. The tubular filamentous material is drawn or expanded axially to create the small diameter configuration that is inserted into the patient. A standoff attaches the distal end of the tubular filamentous material to the sheath hub by way of radially movable anchors. Additional filamentous tubular material extends out the proximal end of the hub. A compression mechanism forces the additional filamentous tubular material in the distal direction which causes axial compression and radial or diametric dilation of the working length of the sheath, that part of the sheath that extends beyond the proximal end of the hub. Radial dilation is accomplished with no substantial change in sheath working length.

### PRIORITY INFORMATION

This application claims priority to U.S. Provisional application Ser. No. 60/569,519, filed on May 7, 2004, titled RADIALLY EXPANDABLE MEDICAL ACCESS SHEATH, the entirety of which is hereby incorporated herein by reference.

### BACKGROUND OF THE INVENTION

1. Field of the Invention

This invention relates to medical devices and techniques, and, in particular to, devices and techniques for accessing and instrumenting surgical sites.

2. Description of the Related Art

Surgical repair can be carried out through open surgical access or through minimally invasive access procedures. Minimally invasive access often involves the creation of one or more small incisions in the skin and then tunneling through the underlying muscle, fascia, and other tissue to reach the target surgical site. A tubular sheath is generally inserted through the tunnel creating a channel through which instruments and monitoring devices may be introduced to the target surgical site. The tubular sheath may be an integral part of a tunneling device. Such a device is often referred to as a trocar. The trocar is inserted through the sheath and is used to sharply, or bluntly, dissect the tissue. When the trocar is removed, the sheath remains in place to provide a clear path for the insertion of instruments for therapeutic or diagnostic purposes. The sheath serves to act as a surgical retractor to keep the tissue out of the way while the procedure is being completed.

Open surgical approaches, by their open nature, require the elements of incision, dissection, hemostasis, and mechanical closure. The incision is typically accomplished using a scalpel, a saw, or an electrosurgical cutting device. Dissection is typically accomplished using a scalpel, electrosurgical cutting device, or a blunt object such as a pair of forceps or an obturator. Hemostasis control is generally performed using electrocautery, wound packing, and suction drainage to a collection system. Tissue removal is generally accomplished with graspers or suction. Mechanical closure is generally accomplished using sutures, staples, or clips. This surgical approach affords direct surgical vision, direct tactile feedback along with the intrinsic ability to enlarge the field of view simply by enlarging the incision and resetting the self retaining retractor.

Many open surgical procedure benefit from the application of self-retaining retractors, dilators, or cannula that provide tissue retraction throughout a surgical procedure without the continued attention of a human assistant. Self-retaining retraction provides the operator with the ability to choose the tissue and separation plane to provide adequate exposure for a given surgical procedure. Retractors commonly known in the surgical art include the Richardson retractor, the Alm retractor, the Balfour retractor, the Rigby retractor and the like.

The advantage of minimally invasive surgery is that the damage to the tissue surrounding the access site is minimized. For example, it is preferable to gently dilate or retract muscle tissue rather than cutting through the muscle tissue. The dilated muscle, once the dilation is removed, returns to its normal function. The severed or cut muscle tissue must be reattached and allowed to heal before it can return to its normal function. The healing may be very long, on the order of weeks or months, and may be accompanied by significant pain and loss of function. Large incisions are also a source of extended patient discomfort and poor cosmesis along with expensive recovery periods, both in and out of the hospital.

Many surgical procedures have been converted to minimally invasive, laparoscopic procedures that avoid large incisions, reduce hospital stays and costs while producing similar short- and long-term results. However, surgical procedures that do not invade body cavities, such as the abdomen or thorax may not be suited for traditional laparoscopic visualization. Such is the case in orthopedic procedures involving joint or spine access. In these cases, a surgeon often is forced to rely on the blunt placement of consecutively larger cannula, with or without benefit of a dilator to reach the desired surgical site. Generally, in these cases, there is no natural cavity or opening at the target surgical site. Surgical instruments are then inserted through the cannula to reach the target site. Surgical exposure is limited by the accurate placement of the cannula, location of pathology and diameter of the cannula. Once the skin incision of adequate size is made, the axial shear force of sequentially placed dilators, with increasing diameters, creates an operative tunnel to reach the desired surgical site.

The rigid walls of the cannula exert a tamponade pressure to provide at least some degree of hemostasis during the procedure. Distal visualization is often provided by a rigid scope while operative maneuvers are accomplished with laparoscopic or extended length instruments placed through the cannula. Radial pressure holds the cannula over the operative site freeing the operative team from retraction duties as well as removing potential obstructive nuisances from the immediate surgical field. Enlarging the surgical field requires placement of one or more progressively larger cannula with an axially directed shear force. Such placement of a new cannula carries with it the possibility of losing anatomic landmarks during the device transition. Since a majority of the tissue plane separation is achieved with blunt expansive force, rather than by tissue shearing, and is maintained with radial force, recovery is often less traumatic than that encountered with open surgery. Should the operative procedure require expansion with incision and traditional retraction applied, the recovery course may be longer and costs higher than if the minimally invasive approach only was used.

Traditional laparoscopy also uses trocars and sheaths, with diameters ranging from 5 to 20 mm, to gain access to the abdomen or chest. Most procedures are successfully completed through three or four trocar sites. There are cases, such as removing an organ or tissue for transplant, when time and labor burden could be reduced by the ability to enlarge a trocar site. Surgical incision sites must be enlarged carefully, however, because most operators are reluctant to expand surgical exposure at the risk of losing the anatomic landmark.

New devices and methods are needed to create a tunnel to a target surgical site in such a way that trauma to the tissue is minimized. These devices need to provide for controllable tissue dilation once the initial tunnel is created. The devices should maintain substantially constant working length so that the physician may be assured of maintaining tissue retraction all the way to the target surgical site, even after dilation. Such devices are particularly important for use in treating lesions of the spine or for cancer therapy, for example. These devices are useful for minimally invasive, least invasive, and percutaneous procedures that may or may not require a cutdown and may or may not be useful in endovascular access procedures.

### SUMMARY OF THE INVENTION

U.S. Pat. No. 5,460,170 to Julius G. Hammerslag, the entirety of which is hereby included herein by reference, discloses an adjustable medical retractor incorporating the elements of a tubular filamentous sheath wherein the filaments are compressed axially to cause the sheath to dilate radially. Conversely, when the filaments of the sheath are expanded longitudinally, the diameter of the sheath becomes smaller. The sheath incorporates a control mechanism at its proximal end. The control mechanism retracts pull wires that are attached to the distal end of the tubular filamentous sheath causing axial compression and radial dilation of the sheath. The pull wires are wound onto a spool located at or near the proximal end of the sheath. This device provides a simple way of dilating a tubular sheath to allow for increased instrumentation and monitoring area. However, because the Hammerslag device shortens considerably in order to transform from its radially compressed to its radially dilated configuration, it is difficult to place the distal end of the sheath and be assured that it will still reach the target surgical site following radial dilation.

Accordingly, one embodiment of the present invention comprises an access sheath for expanding an opening in biological tissue from a first cross-sectional area to a second larger cross-sectional area. The sheath includes a hub having a distal end and a proximal end and an opening extending therethrough. A substantially tubular mesh extends through the hub and comprises a distal portion that extends from the distal end of the hub and a proximal portion that extends proximally from the proximal end of the hub. A radially expandable standoff extends distally from the hub and is coupled to a distal portion of the mesh and to the hub. A compression mechanism is configured to advance the proximal portion of the mesh distally towards the hub causing axial compression and diametrical expansion of the mesh.

Another embodiment of the present invention comprises a method of providing access to a surgical site. In the method, a sheath, which comprises a hub and an expandable tubular member that extends through the hub, is inserted into an incision. A distal portion of the tubular member is advanced to a target depth. The distal portion of the tubular member is expanded diametrically while simultaneously advancing a proximal portion of the tubular member distally toward the hub.

Another embodiment of the present invention comprises surgical access device for expanding an opening in a patient. The device includes a hub having a distal end and a proximal end and an opening extending therethrough. A radially expandable tubular body comprises a distal portion that extends distally of the hub, a proximal portion that extends proximally of the hub, and an axial lumen therethrough. The device further includes means for applying an axially compressive force to the tubular body to reversibly expand the axial lumen from a first, smaller, diameter to a second, larger diameter, without substantially shortening the axial length of the distal portion of the tubular body.

Another embodiment of the present invention comprises a method of providing access to a surgical site which includes providing a sheath having a hub and an expandable tubular member comprising a distal portion and a proximal portion, the distal portion extending distally of the hub and the proximal portion extending proximally of the hub, inserting the distal portion of the tubular member into an incision, advancing the distal portion of the tubular member to a target depth, and expanding the distal portion of the tubular member diametrically without substantially changing the axial length of the distal portion with respect to the hub.

Embodiments of the present invention relate to sheaths, trocars, cannulae, or surgical retractors to be used in minimally invasive surgical procedures in humans or other animals. In one embodiment, the device is a generally tubular or axially elongate, hollow sheath that is inserted into an animal or patient through a small surgically created incision. The incision is created using a cutdown or a percutaneous method such as that known as the Seldinger technique. Once inserted and advanced to the target surgical site, the sheath is controllably expanded to a substantially pre-determined diameter. The wall of the sheath is fabricated from a filamentous tubular structure such as a braid. The filamentous tubular structure is inserted, into the patient, in its axially expanded and radially contracted configuration. The filamentous tubular structure is then contracted or compressed along its longitudinal axis causing the generally axially oriented filaments to become more circumferentially oriented. This reorientation of the filaments causes the sheath to increase its diameter. As the circumferential sheath elements are more tightly packed, thus forming a nearly contiguous array of circumferential filaments with little or no spacing therebetween, the hoop strength of the tubular structure is maximized. In this embodiment, the device includes a structure that maintains a substantially constant distance, defined as working length, between the distal end of the sheath and the part of the sheath that is handled by the physician, herein referred to as the hub.

In one embodiment of the invention, a sheath comprises a handle or hub, which is grasped by the user. The hub further incorporates radially expandable attachment points, which are attached to a plurality of standoffs, which project out the front or distal end of the hub. The standoffs extend substantially all the way to the distal end of the sheath. The standoffs are coupled to a tubular structure composed of filamentous elements, which are wound or braided to extend along the axial length of the tubular structure but which further are biased to at least partially be disposed in the circumferential direction. Compression of the tubular structure from the proximal end of the tubular structure toward the distal end of the tubular structure causes the tubular structure to compress along its longitudinal axis and expand in a direction perpendicular to the longitudinal axis, defined herein as the radial direction. The standoffs provide a counterforce against which the tubular structure is compressed and maintain the working length of the sheath at a constant, pre-determined length. The standoffs further serve to enhance or increase the resistance to deformation of the sheath. The radially expandable attachment points allow the tubular structure to dilate but constrain the standoffs axially within the hub. Various embodiments of compressing the tubular structure toward the distal end of the device, include rams, pull wires, pinch rollers, jackscrews, shape memory contraction, and the like.

In one embodiment of the present invention, excess sheath material is provided. The excess material is to be compressed axially in order to radially dilate the working length of the sheath as is drawn from the proximal end of the sheath, in most cases, proximal to the hub. A mechanical lock can be provided in the hub to allow the dilation control element to be selectively constrained, or unconstrained, and thus lock the sheath diameter in place. The sheath is preferably supplied with an internal obturator, a proximal seal for instruments and for providing hemostasis, and insulation or an expandable barrier layer to prevent or minimize the escape of energy, fluids, or contaminants from the interior of the sheath to surrounding tissue.

In one embodiment of the present invention, the sheath includes a guidewire channel, either through the sheath itself or through the center of a removable obturator. This guidewire channel provides the ability to insert the sheath, in its small diameter configuration, over a guidewire. In another embodiment, the sheath can be used as a probe under radiographic guidance (fluoroscopy, computer aided tomography (CAT), magnetic resonance imaging (MRI), or ultrasound). In the fluoroscopic version, the sheath may include radiopaque markers that provide for enhanced visualization under X-ray or fluoroscopic observation, even with a background of dense tissue and bone. The sheath can further be inserted and manipulated under direct vision by including a small caliber endoscope to identify an anatomic path or features within a body cavity.

In one embodiment of the present invention, once initial tissue target is identified and the appropriate location of the access confirmed, the device can, under direct, precise operator control, enlarge the access lumen by applying radial force. The surrounding tissue applies a counter pressure to that exerted by the radially dilated device, which aids in maintaining stability of the device once it is expanded. The overall diameter of the sheath can be reduced, at operator discretion, by first unlocking and then reversing the procedure used to axially compress the tubular element. This mode of maintaining surgical retraction can be termed passive.

In another embodiment, which is different than the passive mode of maintaining surgical retraction, the device can be introduced via standard laparoscopic trocar and be expanded to stabilize an organ or tissue with known, controlled circumvention pressure to provide the operator with a stable operative surface. Additionally, the device can be positioned to displace organs and structures to create a stable tunnel to expose a distant operative site.

In yet another embodiment, a laparoscopic version of the sheath is used to create a dome over the surgical site. The dome, or end enlargement, is created at the distal end of the sheath and is preferably radially dilated separately from the sheath. The dome is, in this embodiment, a separate tubular structure of filamentous material with different expansion characteristics than those of the main sheath. Compression of both structures can occur at once. In another embodiment, the dome could be axially compressed and radially expanded separately from the main sheath if it were positioned distal to the attachment point of the standoff. In this embodiment, pull wires could be used to separately dilate the dome following main sheath tube dilation or expansion. An elastomeric, expandable, or unfurling material connecting the delivery sheath to the dome provides a seal at the proximal end of the dome to isolate tissues captured within the dome from surrounding tissues. The exterior of the device would gently move uninvolved organs and tissue out of the surgeon's area of interest. The interior of the dome would provide adequate space for visualization, sewing and clipping for hemostasis as well as for tissue re-approximation. Infusion and removal of fluids are preferably enabled by access ports and lumens on the sheath and made available to maintain a clear surgical field.

Another embodiment of the present invention involves a method of use wherein the device is inserted as part of a system to capture an organ. The sheath is inserted to allow safe withdrawal of another device designed to contain the amputated organ or tissue, which can then be withdrawn through the sheath to a position outside that of skin level. This method conveys the benefit of laparoscopic surgery while avoiding the challenges associated with isolated removal of a diseased organ where malignant cell isolation is of a concern.

In accordance with embodiment of the present invention, a diseased organ or damaged tissue mass is isolated by the surgeon by inserting the sheath to the target site. The sheath is then dilated to generate a larger diameter working channel than that of the originally inserted instrument. During the radial dilation, the working length of the sheath is held constant so that the sheath continues to reach the target site. In order to maintain a constant working length, the extra sheath material needed to generate a diameter increase is routed from a source proximal to, or internal to, the region where the user grasps the sheath. An instrument can then be inserted through the sheath. These instruments may allow for various methods of cell or tissue destruction to be employed, with or without specimen removal. Access to the diseased organ may be accomplished under direct vision as part of a laparoscopic or percutaneous procedure. Exemplary uses of a sheath that may be selectively enlarged include applications in procedures to remove kidney stones, perform biopsies or organ removal, or perform implantation of spinal devices. The sheath may further be used to insert devices to repair damaged orthopedic joints, perform laminectomies, and the like. Following completion of the procedure, the sheath is removed from the patient, with or without the step of reducing the size of the sheath before removal.

In another embodiment of the present invention, an insulating barrier placed on the outside, or inside, of the filamentous tubular structure would confine therapeutic or diagnostic cryogenic temperatures, radio frequency (RF) waves, or microwaves so that they would not substantially reach tissues surrounding the sheath. Instead of sustaining losses along the length of the sheath, these energies are focused substantially on the tissue or organ targeted by the device at or near its distal end. In another embodiment, a seal layer is provided that serves as a barrier and prevents migration of fluids and other materials through the wall of the sheath. The seal layer, also called a containment layer, may be either elastomeric, it may be inelastic but unfurling, in nature, or both. An insulating exterior or interior barrier that protects displaced, healthy tissue from destructive treatments being applied to diseased tissue within the confines of the device. Electrical, thermal and radiated options should be incorporated. Tissue treated in this manner could be desiccated and rendered inert and of a reduced size for more easy removal. Furthermore, healthy tissue outside the sheath is protected against contamination by pathological tissue being removed or accessed by the sheath. Such protection of healthy tissue is especially important in the case of malignant or carcinogenic tissue being removed through the sheath so that potential spread of the disease is minimized.

For purposes of summarizing the invention, certain aspects, advantages and novel features of the invention are described herein. It is to be understood that not necessarily all such advantages may be achieved in accordance with any particular embodiment of the invention. Thus, for example, those skilled in the art will recognize that the invention may be embodied or carried out in a manner that achieves one advantage or group of advantages as taught herein without necessarily achieving other advantages as may be taught or suggested herein.

These and other objects and advantages of the present invention will be more apparent from the following description taken in conjunction with the accompanying drawings.

### BRIEF DESCRIPTION OF THE DRAWINGS

A general architecture that implements the various features of the invention will now be described with reference to the drawings. The drawings and the associated descriptions are provided to illustrate embodiments of the invention and not to limit the scope of the invention. Throughout the drawings, reference numbers are re-used to indicate correspondence between referenced elements.

FIG. 1A illustrates a side cross-sectional view of an embodiment of a radially expandable sheath in a first, reduced profile configuration;

FIG. 1B illustrates the sheath of FIG. 1A in a second, expanded profile configuration;

FIG. 2A illustrates a side cross-sectional view of another embodiment of a radially expandable sheath in a first, reduced profile configuration;

FIG. 2B illustrates the sheath of FIG. 2A in a second, expanded profile configuration;

FIG. 3A illustrates the sheath of FIG. 2B without a compression element;

FIG. 3B illustrates a front view of a cam lock according to an embodiment of the invention;

FIG. 4A illustrates an another embodiment of a radially expandable sheath in a first, reduced profile configuration;

FIG. 4B illustrates the sheath of FIG. 4A in a second, expanded profile configuration;

FIG. 4C is a side view of an embodiment of an obturator;

FIG. 5A is a lateral view of another embodiment of radially expandable sheath in a first, reduced profile configuration;

FIG. 5B illustrates a lateral view of the sheath of FIG. 5A in a second, expanded profile configuration;

FIG. 6A illustrates a side view of another embodiment of a radially expandable sheath in a reduced profile configuration;

FIG. 6B illustrates the sheath of FIG. 6A in an enlarged profile configuration;

FIG. 7A illustrates a close-up view of fibers or filaments of a mesh in an axially elongated and radially compressed state, according to an embodiment of the invention;

FIG. 7B illustrates a close-up view of the mesh of FIG. 7A wherein the fibers or filaments of the mesh are now axially compressed and radially dilated, according to an embodiment of the invention;

FIG. 8A is a side view of another embodiment of an expandable sheath comprising its minimum diameter configuration;

FIG. 8B is a side view of the sheath of FIG. 8A its maximum diameter, configuration;

FIG. 9 is a side view of another embodiment of a radially expandable sheath which further comprises a dome at its distal end;

FIG. 10A is a side view of two ends of a braided sheath structure in a first position;

FIG. 10B is a side view of the ends of the braided sheath structure op FIG. 10A in a second position;

FIG. 10C side view of the ends of ta braided sheath structure, further comprising a flexible coil hinge; and

FIG. 10D is a schematic illustration of four ends of at the distal end of an embodiment of a sheath.

### DETAILED DESCRIPTION OF THE PREFERRED EMBODIMENTS

In the embodiments described herein, reference will be bade to a catheter or a sheath. A catheter or sheath can generally be described as being an axially elongate hollow tubular structure having a proximal end and a distal end. The axially elongate structure further has a longitudinal axis and has an internal through lumen that extends from the proximal end to the distal end for the passage of instruments, implants, fluids, tissue, or other materials. The axially elongate hollow tubular structure can be rigid or it can be generally flexible and capable of bending, to a greater or lesser degree, through one or more arcs in one or more directions perpendicular to the main longitudinal axis.

As is commonly used in the art of medical devices, the proximal end of the device is that end that is closest to the user, typically a surgeon or interventionalist. The distal end of the device is that end closest to the patient or that is first inserted into the patient. A direction being described as being proximal to a certain landmark will be closer to the surgeon, along the longitudinal axis, and further from the patient than the specified landmark. The diameter of a catheter is often measured in “French Size” which can be defined as 3 times the diameter in millimeters (mm). For example, a 15 French catheter is 5 mm in diameter. The French size is designed to approximate the circumference of the catheter in mm and is often useful for catheters that have non-circular cross-sectional configurations. While the original measurement of “French” used pi (3.14159 . . . ) as the conversion factor between diameters in mm and French, the system has evolved today to where the conversion factor is exactly 3.0.

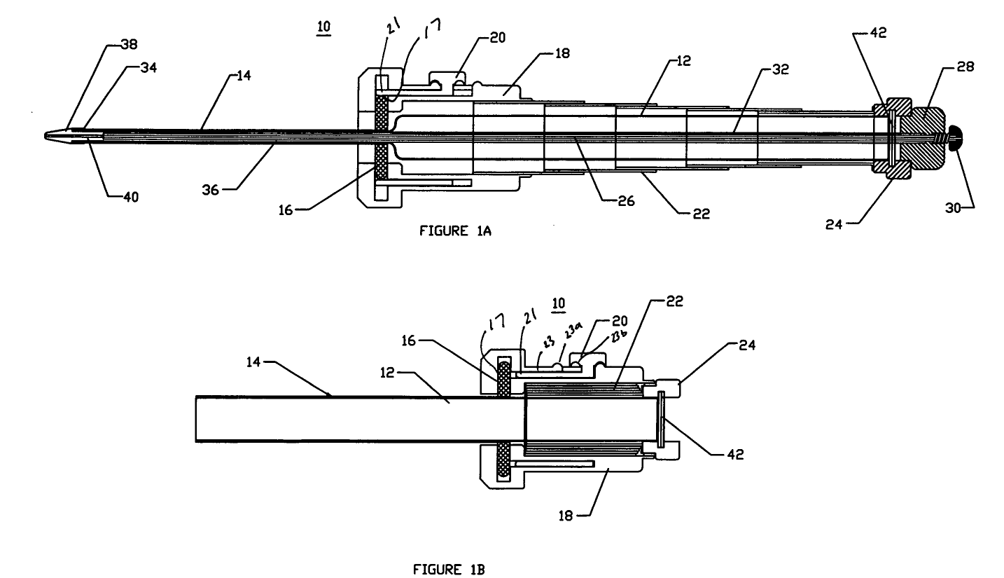

FIG. 1A illustrates a lateral cross-sectional view of an a first embodiment of an expandable sheath 10 in its radially compressed configuration. In the illustrated embodiment, the expandable sheath 10 comprises a tubular mesh 12, at least one standoff 14, at least one anchor 16, a hub 18, an anchor lock 20, a telescoping ram 22, a seal hub 24, an obturator 26, an obturator handle 28, and an obturator button 30. The obturator 26 further comprises an obturator shaft 32, a sleeve 34, an obturator control rod 36, an obturator nose cone 38, and an obturator guidewire lumen 40. These components will all be described in detail below.

With initial reference to FIG. 1A, the hub 18 serves as a point of stability and user grip for the sheath 10. The anchor or anchors 16 are coupled to the hub 18, within a cavity 17 such that they slide or move in the radial direction but are constrained not to move substantially in the direction parallel to the longitudinal axis of the sheath 10. The proximal ends of the standoffs 14 are coupled to the radially innermost aspect of the anchors 16. The distal ends of the standoffs 14 are coupled to the distal end of the tubular mesh 12. The proximal end of the hub 18 is releasably coupled to the seal hub 24 through the telescoping tube 22. Preferably, the telescoping tube 22 is constrained to move somewhat within the proximal end of the hub 18 but is not capable of being disconnected from the hub 18.

As shown in FIG. 1A, segments of the telescoping tube 22 are capable of being extended in the direction of the longitudinal axis, proximally away from the hub 18. The distal most portion of the distal most segment of the telescoping tube 22 is coupled to the seal hub 24. When the telescoping tube 22 is fully compressed axially (see FIG. 1B), the seal hub 24 mates with, and is preferably releasably, controllably locked to the proximal end of the hub 18.

With continued reference to FIG. 1A, the proximal end of the tubular mesh 12 is coupled to the seal hub 24 or, in a modified embodiment, the distal most segment of the telescoping tube 22. The seal hub 24 further comprises a seal 42 that is coupled to the inner aspect of the seal hub 24. The obturator 26 is further comprised of an obturator handle 28 and an obturator button 30. The obturator 26 is slidably movable within the tubular mesh 12 and is removable from the sheath 10. The obturator handle 28 is on the proximal end of the obturator 26 and is releasably coupled to the proximal end of the seal hub 24. The obturator button 30 is constrained to move within predefined limits about the obturator handle 28. Depression or pulling motion on the obturator button 30 causes a sleeve 34 at the distal end of the obturator 26 to disconnect from the distal most part of the mesh 12 so that the obturator 26 can be removed.

The tubular mesh 12 can be fabricated from materials such as, but not limited to Elgiloy, nitinol, titanium, polytetrafluoroethylene (PTFE), stainless steel, polyamide, polyester, and the like. In the illustrated embodiment, the tubular mesh 12 is formed from structures such as a tubular braid, a counterwound spiral spring, a single spiral spring, and the like. The tubular mesh 12 may further be coated on one or both sides with anti-thrombogenic agents such as, but not limited to, heparin, which is ionically, or covalently, bonded to the tubular mesh 12. The tubular mesh 12 may also be coated with anti-microbial agents such as, but not limited to, silver oxide, silver azide, betadine, povidone iodine, or the like. The tubular mesh 12 may further be configured to carry electrical charge so that it can be used as the electrode for microwave or radio frequency (RF) energy, which can be used to cauterize or destroy cellular tissue.

Exemplary construction for the tubular mesh 12 illustrated in FIG. 1A is a braid comprising 2 ends per carrier and 24 carriers braided over a 0.375 inch diameter mandrel. The number of ends per carrier could range between 1 and 10 while the number of carriers could range between 2 and 50. In one embodiment, the filaments are 0.015-inch diameter round or 0.008 by 0.011 rectangular flat wire. The individual filament diameters or rectangle side dimensions may range from 0.001 to 0.040 inches. The wall thickness of the tubular mesh 12 ranges from about 0.0002 inches to about 0.060 inches and is preferably between 0.010 and 0.040 inches. The tubular mesh 12 is preferably configured so that it may be easily, with little applied force required, compressed, radially to less than 0.25 inches diameter and may be fully expanded to approximately 0.90 inches in diameter. The working length of the tubular mesh 12 is determined by the distance between the skin surface and the target surgical site. Typical working lengths range from 1 cm to 150 cm and more typically range between 5 cm and 30 cm. The working length is that distance between the distal most edge of the tubular mesh 12 and the distal end of the hub 18. The tubular mesh 12 projects axially into and through the inner lumen of the hub 18 but this part proximal to the distal end of the hub 18 is not useable length for cannulating a patient. The radial, or hoop, strength of the tubular mesh 12 in the described configuration is sufficient to expand most soft tissue in a uniform circular fashion. This radial strength is achieved by axially compressing and stacking successive filaments of the tubular mesh 12 to create a structure that has resistance to inward hoop stress and point loads. The tubular mesh 12 further comprises an internal through lumen for passage of instruments, materials and the like, when it is integrated into the sheath 10.

In one embodiment, the distal edge of the tubular mesh 12 is not edge treated in any way. In another embodiment, the distal edge of the tubular mesh 12 is embedded in, or surrounded by, an elastomeric membrane that helps control the distal ends of the filaments. In this embodiment, the distal edge of the tubular mesh 12 is controlled by coating with a polymer, or other technique known in the medical art. In yet another embodiment, the distal edge of the tubular mesh 12 comprises filaments or groups of filaments, preferably flexible, that are welded together using methods such as, but not limited to, heat, solvents, adhesives, or ultrasonic energy. This technique fixes the angle between filaments, which means that a change in diameter at the distal end of the sheath mesh 12 would impart bending stresses on the weld or the filaments adjacent thereto. In yet another embodiment, the distal edge of the tubular mesh 12 comprises small hinges (not shown) that permit the ends of the individual filaments to be coupled to adjoining filaments. The hinges permit angular rotation of one filament relative to another connected filament without excess or undue force, which would be required if the ends were welded at a fixed angle. In another embodiment, each of the ends of the individual filaments of the mesh 12 are configured with a coil that subtends an angle equal to or greater than 180 degrees, and most likely greater than 270 degrees. The coil can have a second or third turn, or even more turns, equal to 360 degrees per additional turn. The coil (not shown) permits rotation of one end relative to another and minimizes the stresses on the filaments when the relative angle changes. The coil can be made from the same wire as the filament or it can be a separate wire. Control of the distal edge of a braided sheath is important since loose ends can cause skin cuts. One end of the coil is coupled to one filament and the other end of the coil is coupled to an adjacent filament. In another embodiment, a ring or sleeve of elastomeric polymer encases the distal edge of the mesh 12. In another embodiment, a ring or sleeve of elastomeric polymer is disposed on the exterior of the distal edge of the mesh 12 and extends slightly beyond the distal edge of the filaments. The ring or sleeve of elastomeric polymer is preferably of very low durometer, for example Shore 5A to Shore 75A, and is substantially relaxed at the small diameter of the sheath 10. The ring, sleeve, or coating can be fabricated from silicone elastomer, latex rubber, thermoplastic elastomer, polyurethane elastomer, or the like. It is beneficial that the ring or sleeve not fill the spaces between the filaments of the mesh so that the filaments can move and rotate relative to each other as the mesh expands and contracts.

The mesh 12 is preferably configured so that it is pre-compressed axially in the region proximal to the anchors 16. By pre-compressing at least part of the mesh 12, it is possible to reduce much of the excess material length needed in order to achieve a desired radial expansion of the working length of the sheath 10. It is possible to maintain the pre-compression by affixing tensioning cables between the proximal end of the mesh 12 and a region located at least somewhat proximal to the anchors 16. Tensioning cables (not shown) can be, for example, sutures or threads tied between the aforementioned points. The cables can be routed outside, inside, or woven inside and outside the mesh 12 as they are routed between the attachment points. The pre-compression can also be accomplished by adhering, welding, encapsulating, or bonding the filaments of the mesh 12 in the pre-compression area. Pre-compression of the mesh 12 in the region distal to the anchors 16 defeats the ability to expand and compress the mesh 12 in the radial direction. The pre-compressed region of the mesh 12 can, in another embodiment, be a non-compressing, non-expanding tubular structure fabricated from materials the same as, or completely different from those of the mesh 12. By way of example, a mesh 12 that expands diametrically from 6 mm to 20 mm with a working length of 15 cm requires about 48 inches of material length. Pre-compression permits the overall length of the mesh 12, and therefore the sheath 10, to be reduced from 48 inches to less than 17 inches.

The standoff 14 is preferably fabricated from materials such as, but not limited, to polyester, polyamide, stainless steel, Elgiloy, nitinol, and the like. The standoff 14 is preferably sized so that its length is slightly longer than that of the desired sheath 10 working length. The length of the standoff 14 is preferably between 1-cm and 50-centimeters. The standoffs 14 may be configured to be wire, bars of triangular, rectangular, or other geometric cross-section, or they may be configured as a cylinder that is slit along its longitudinal axis to form a plurality of bars with partial cylindrical cross-sections. The standoff 14 is preferably spring hardened, either by heat-treating or cold working, although a malleable, non elastomeric standoff is suitable for certain applications. By configuring the standoffs 14 as parts of a cylinder, in the radially compressed configuration, the standoffs 14 can form a contiguous cylinder surrounding an internally disposed mesh 12. The curved partially cylindrical shape of a standoff 14 offers advantages in terms of improved rigidity, relative to non-curved cross-sectional shapes. The standoff 14 may be perforated at its distal end for attachment to the mesh 12 using sutures, wires, welding, bonding, or the like. The standoff 14 may be attached to the anchor 16 using a screw or other fastener, it may be integral to the anchor 16, or it may be insert molded into the anchor 16. There is preferably one anchor 16 for each standoff 14. The number of standoffs 14 and anchors 16 can range from 1 to 30 with a preferred range of 2 to 10. The anchors 16 are preferably fabricated from metals such as stainless steel, nitinol, Elgiloy, or the like. The anchors 16 may further be fabricated from hard or lubricious polymers such as, but not limited to, polyacetal, polypropylene, polyethylene, polytetrafluoroethylene, polyester, polycarbonate, polysulfone, or the like. The standoff 14 further serves to increase the rigidity of the mesh 12 against radially directed forces and distributes applied stresses longitudinally along the mesh 12.

The hub 18, the lock 20, and the seal hub 24 are preferably fabricated from materials such as, but not limited to, acrilonitrile butadiene styrene (ABS), polyvinyl chloride (PVC), polyethylene, polypropylene, and the like. The seal 28 is preferably fabricated from a soft elastomer such as, but not limited to, silicone elastomer, latex rubber, thermoplastic elastomer such as C-Flex, polyurethane, and the like. The seal hub 24 is preferably reversibly coupled to the hub 18 using a bayonet mount, screw thread, a releasable latch, or the like. By locking the seal hub 24 to the hub 18, the filaments of the mesh 12 are compressed axially and maintained in that configuration so that the maximum diameter possible is sustained by the sheath 10. When the seal hub 24 is released or unlatched from the hub 18, the filaments of the mesh 12 may be pulled longitudinally or relaxed to cause a reduction in the diameter of the working length of the sheath 10.

The lock 20 is, in one embodiment, a positive lock that is engaged to prevent, or disengaged to allow, relative radial rotation between the anchors 16 and the hub 18. In another embodiment, the lock 20 is a positive cam or rotational lever such as that found in a collet on a lathe. In this later embodiment, the lock 20 is rotated to force the anchors 16 to move either radially inward or outward. A ramp, or series of ramps, integral to the lock 20, engages slots in the anchors 16 and forces the anchors to move radially inward or outward when the lock 20 is rotated. The lock 20 may further comprise a ratcheting mechanism that permits rotation or release of the lock 20 with binding or frictional click-stops. The lock 20 is further configured not to require the attention of an assistant to maintain position. In another embodiment, an outer fixture may be placed at skin level and selectively affix to the hub 18 to assist with stabilization. In the illustrated embodiment of FIGS. 1A and 1B, the lock 20 includes an arm 21 slidably positioned within a opening 23 in the hub 18. The lock 20 includes a tab with a recess 23b that engages a protrusion 23a on the hub. As shown, by disengaging the recess 23b with the protrusion 23a and proximally retracting the lock the arm allows the anchors 16 to radially expand within the groove 17.

The anchors 16 are, in the illustrated embodiment, axially elongate rods of rectangular, circular, or other geometric cross-section that ride within grooves, slots, tracks, or channels 17 in the hub 18. The longitudinal axis of the anchors 16 and the grooves within the hub 18 are disposed generally perpendicular to the long axis of the sheath 10 so that the anchors 16 move in a generally radial direction. The movement of the anchors 16 within the hub 18 is preferably accompanied by low friction so that the anchors 16 can adjust radial position within the hub 18 to accommodate dilation of the mesh 12 and the standoffs 14. In another embodiment, the anchors 16 are torsion bars that are coupled within the hub 18 and simply bend to hold the proximal end of the standoff 14 in place relative to the hub 18. In this embodiment, there is some minor relative motion parallel to the longitudinal axis of the sheath 10, which accompanies the radial motion, but this longitudinal motion is limited to a substantially small percentage of the total working length of the sheath 10. For example, a bending type anchor 16 might move 2 to 5 millimeters relative to a total working length of 10 to 15 cm, a worst-case scenario of 5%. In this embodiment, the longitudinal movement of the anchor 16 will always be less than 10% of the working length of the sheath 10. In other embodiments, the anchor 16 may be a crankshaft with hinges at the hub 18, the standoff 14, or both, and will operate similarly to the bending anchor 16 but with less friction and more complexity. In yet another embodiment, the anchor 16 is similar to a jack that has diamond-shaped supports with hinges and is constrained to move only in the radial direction. This embodiment would permit substantially little or no longitudinal motion when the anchor 16 moves radially.

The telescoping tube 22 is preferably fabricated from materials such as, but not limited to polycarbonate, glass filled polycarbonate, stainless steel, cobalt nickel alloys, titanium, nitinol, acrilonitrile butadiene styrene, polyvinyl chloride, polytetrafluoroethylene, fluorinated ethylene propylene (FEP), and the like. In this embodiment, the telescoping tube 22 serves as a collapsible connector between the hub 18 and the seal hub 24. A further advantage of the telescoping tube 22 is to restrain the proximal end of the mesh 12 from bending out of the general axis of the sheath 10 and possibly becoming distorted, buckled, or otherwise dysfunctional. While this embodiment shows the telescoping tubing 22 disposed outside the mesh 12, it is possible to place the telescoping restraint within the mesh 12 while still restraining the mesh 12 from lateral motion and permitting controlled longitudinal collapse of the mesh 12.

The obturator 26 further comprising the obturator handle 28 and the obturator button 30 is shown in place within the sheath 10. The distal end of the obturator 26 is controllably, and reversibly, coupled to the distal end of the sheath 10. The proximal end of the obturator 26 is controllably, reversibly coupled to the proximal end of the sheath 10 in the region of the seal hub 24. The obturator 26 further comprises a shaft, not shown, that provides column strength for the obturator 26 and maintains the positioning and distance of the distal tip relative to the obturator handle 28, which is coupled to the proximal end of the obturator 26. The obturator shaft may be rigid, or it may be flexible to permit bending of the obturator 26 and the working length of the sheath 10 when so that the sheath 10 may be negotiated through somewhat tortuous anatomies during placement.

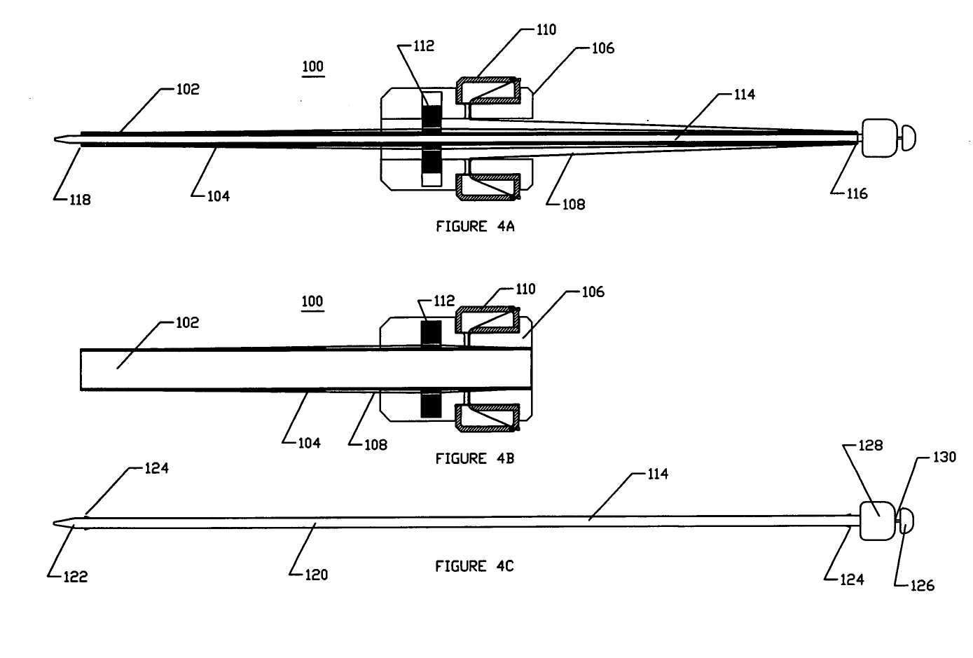

The obturator shaft 32, the obturator nose cone 38, and the obturator handle 28 both preferably comprise a guidewire channel or lumen 40, extending axially from the proximal end to the distal end of the obturator 26, that permits passage of a guidewire (not shown) therethrough. The guidewire channel 40 is generally between 0.005 and 0.100 inches in diameter, with a preferred range being between 0.020 and 0.050 inches. The guidewire channel is preferably tapered at the proximal end to permit easier insertion of a guidewire and it preferably comprises a Tuohy Borst fitting or other valve or seal (not shown) to resist the leakage of fluids from the guidewire channel. The valve seals the annulus that is created between the guidewire and the inner diameter of the channel 40 through which the guidewire passes. The obturator button 30 permits activation of a mechanism within the obturator 26 that releases the distal end of the obturator 26 from the sheath 10 and optionally releases the obturator handle 28 from the seal hub 24. In a preferred embodiment, the sleeve 34 at the distal end of the obturator 26 or retractable prongs (item 124 FIG. 4C) permit gripping of the sheath 10 at its distal end by the obturator 26. Depression of the obturator button 30 moves the control rod 36, to which the button 30 is coupled, that, in turn, moves the sleeve 34, to which the control rod 36 is coupled, so that the sleeve 34 no longer covers the sheath 10 distal end and permits release of the obturator 26 from the sheath 10. In a preferred embodiment, the control rod 36 is concentric to and is constrained to move axially only by the obturator shaft 32, which is coupled to the obturator handle 28. It is desirable to release the obturator 26 from the sheath 10 prior to expansion of the sheath 10 using the means described herein. The obturator 26 further comprises a nose cone 38 or other blunt, sharpened, or faired geometry that facilitates placement of the sheath 10 into tissue without causing unwanted tissue damage. In another embodiment, the sleeve 34 is replaced with spikes, hooks, or barbs that grab the radially collapsed sheath 10 from the inside to hold the obturator 26 in place. Depression of the button 30 relative to the handle 28 moves the control rod 36 that retracts the barbs or hooks (shown in FIG. 4C) to release the obturator 26 from the sheath 10.

The obturator 26 is typically fabricated from materials such as, but not limited to, stainless steel, polymers such as ABS, PVC, polyester copolymers, and the like. The obturator 26 and the entire sheath 10 are fabricated preferably from materials that can be sterilized by methods such as, but not limited to, ethylene oxide, gamma irradiation, electron beam irradiation, steam sterilization, and the like. The obturator 26 and sheath 10 are preferably packaged in a single or double aseptic package that is sealed and are preferably sterilized prior to use.

FIG. 1B illustrates the sheath 10 of FIG. 1A, wherein the seal hub 24 has been advanced distally, relative to the hub 18, and has been couple to the proximal end of the hub 18. In this configuration, the telescoping tube 22 has been axially compressed to its minimum length and the mesh 12 has been fully longitudinally compressed. In this configuration, the mesh 12 has achieved its full radial expansion throughout its entire length. The anchors 16 have moved axially outward within the groove 17 to enable the larger through bore and move the standoffs 14 outward to permit mesh 12 dilation. The arms 21 of the lock 20 has been retracted to allow this movement. During the longitudinal or axial compression, the telescoping tube 22 has restrained the mesh 12 from lateral motion or buckling and has permitted the mesh 12 to be longitudinally compressed without becoming distorted, a situation that could lead to functional failure of the sheath 10. In this configuration, the sheath 10 now has a maximum through bore and is fully dilated along its entire length, now foreshortened compared to its configuration in FIG. 1A. However, the length of the device 10 distal to the hub 18 remains substantially the same as at least a portion of the mesh 12 is advanced through the hub 18. In particular, the standoffs 14 provide a counterforce against which mesh 12 is compressed and maintain the working length (e.g., the portion distal to the hub 18) of the mesh 12 at a constant, pre-determined length. The seal hub 24 permits instruments to be passed into and through the sheath 10. The system can fully sealed against fluid escape by the seal within the seal hub 24. The obturator 26, shown in FIG. 1A, has been removed from the sheath 10 prior to axial compression.

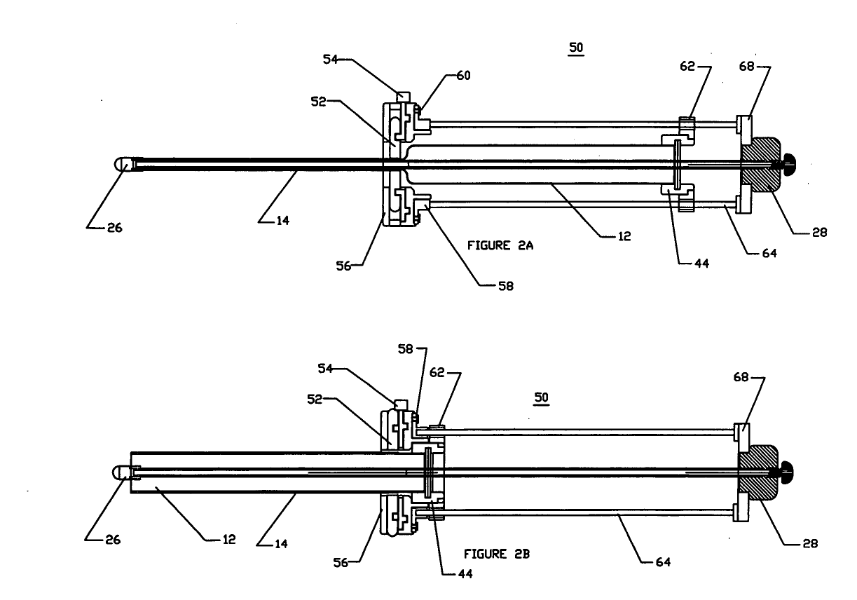

FIG. 2A illustrates an other embodiment of an expandable sheath 50 that comprises the mesh 12, the plurality of standoffs 14, the seal hub 44, the obturator 26 further comprising the obturator handle 28, a plurality of cam anchors 52, a cam lock 54, a hub 56, a slide base 58, a bayonet mount 60, a plurality of rails 64, a rail slide 62, and a rail grip 68.

With reference to FIG. 2A, the mesh 12 is coupled at its distal end to the distal end of the standoffs 14. The proximal end of the obturator 26 comprises, and is copuled to, the obturator handle 28. The obturator handle 28 is coupled to the rail grip 68. Preferably the rail slide 62 releasably engages the seal hub 44 so that it is capable of advancing the seal hub 44 distally but disconnects easily from the seal hub 44 when pulled proximally. The rail slide 62 is constrained to move in a direction parallel to the longitudinal axis of the sheath 50 between the limits of the slide base 58 and the rail grip 68. The slide base 58 preferably is reversibly and removably coupled to the hub 56 by threads, pins, bayonet mounts, clips, or the like. The seal hub 44 is capable of catching on the hub 56 and becoming releasably secured in attachment to the hub 56. The rails 64 are coupled to the slide base 58 and the slide grip 68. The cam anchors 52 are coupled, at their radially innermost aspect, to the standoffs 14. The cam anchors 52 are constrained to move with little or no resistance, within the hub 56 along radially disposed slots, grooves, slides, or the like, which are preferably with the hub 56. The cam lock 54 is constrained to rotate within the hub 56 and features on the cam lock 54 engage features on the cam anchors 52 to force the cam anchors radially outward or inward when the cam lock 54 is rotated circumferentially, relative to the axis of the sheath 50. The mesh 12 and the standoffs 14 are at their smallest diameter in the working length of the sheath 50 distal to the distal most edge of the hub 56. The mesh 12 is preferably compressed axially in the region proximal to the cam anchors 52 to minimize excess material protrusion beyond the proximal end of the hub 56.

The mesh 12, and the standoffs 14, of FIG. 2A are substantially identical to, and serve the same function as, those of FIG. 1A. Referring to FIG. 2A, the hub 56 is configured to allow handling of the sheath 50 but is also capable of holding a rotational cam lock 54. The seal hub 44 is configured to reversibly attach to the hub 56 by way of a bayonet mount, snap mount, latch, or other mechanism that permits simple compression attachment and latching. The bayonet mount can require a small amount of circumferential rotation on the part of the seal hub 44, relative to the hub 56. The rail slide 62 is configured to reversibly engage the seal hub 44 so that the rail slide 62 and seal hub 44 can be pushed distally to compress the mesh 12. Following latching of the seal hub 44 to the hub 56, the rail slide 62 is withdrawn proximally, in a direction parallel to the long axis of the sheath 50. The slide base 58 attaches directly to the proximal end of the hub 56 and is removable by way of mounts such as the bayonet mount 60 shown in FIG. 2A. The obturator 26, which can be coupled to the slide grip 68, serves the function of maintaining the mesh 12 constrained generally along the long axis of the sheath 50. The obturator 26 is preferred in this embodiment because unlike the telescoping tubing 22 of FIG. 1A in the sheath 50 there is nothing constrain the mesh 12 from bending out of the general axis of the sheath 50. The obturator 26, being coupled at the proximal and distal end of the sheath 50 provides column strength and permits the sheath 50 to be advanced under compression without changing the diameter of the working length, until the obturator 26 is removed. Again, the obturator 26 may be either rigid or flexible but should have significant column strength. The obturator handle 28 may either be bonded to the slide grip 68, reversibly connected such as with a snap or clip, or it may be integrally formed with the slide grip 68.

FIG. 2B illustrates the sheath 50 of FIG. 2A wherein the rail slide 62 and the seal hub 44 have been advanced distally relative to the sheath 50 and latched to the hub 56. In this configuration, the working length of the sheath 50 is now enlarged diametrically to its maximum value and the working length is substantially unchanged from that of FIG. 2A. As shown, a substantial portion of the mesh 12 that was previously proximal to the hub 56 is now distal to the hub 56. The obturator 26 is now free from the mesh 12 and the standoffs 14 and is free to be removed from the sheath 50 at any time. The rails 64, the slide grip 68, the obturator 26, the obturator handle 28, the slide base 58, and the rail slide 62 are still coupled to the hub 56 in this illustration. The anchors 52 have been withdrawn radially outward by rotation of the lock 54 to allow clearance of the standoff 14 attachment points to the anchors 52 which are axially trapped within the hub 56. In another embodiment, the seal hub 44 may be a Tuohy Borst valve or other annular seal that completely prevents the escape of fluids, even those fluids pressurized to systemic blood pressure, between the seal hub 44 inner diameter and the outer wall of an instrument or catheter passed through the sheath 50. This type of system is especially useful in endovascular applications where the sheath 50 is inserted into the cardiovascular circulation of a patient. In this embodiment, the sheath 50 further comprises a containment layer (not shown), which prevents the escape of fluid through the walls of the sheath 50.

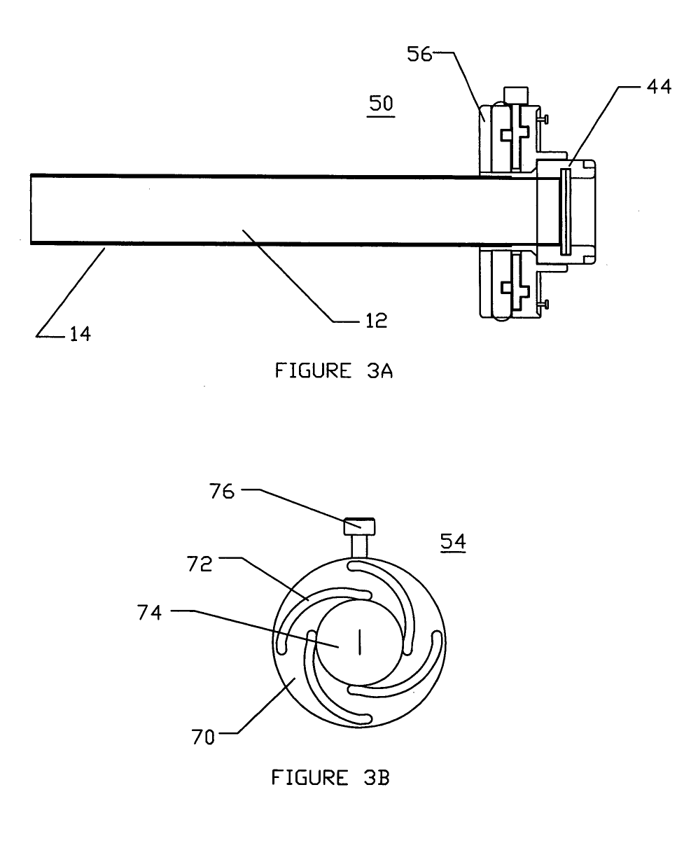

FIG. 3A illustrates another embodiment sheath 50 of FIGS. 2A and 2B wherein certain components have been removed to achieve a sheath 50 with less protrusion axially beyond the hub 56. Specifically, in this embodiment, only the seal hub 44 remains protruding or extending proximally beyond the hub 56. In addition, only the standoffs 14 and the mesh 12 remain protruding beyond the distal end of the hub 56. The hub 56 further comprises optional attachment geometry to facilitate attachment of devices such as cameras, light sources, therapeutic devices, and the like. The hub 56 further may comprise optional components to affix the sheath 50 to the patient. Such fixation components include flanges for tape, straps, or other hold-down devices.

FIG. 3B illustrates a front view of the distal side of the cam lock 54 which further comprises a base plate 70, a plurality of ramps 72, a central through orifice 74, and a handle 76. The cam lock 54 is preferably fabricated integrally from materials such as, but not limited to, stainless steel, polyacetal, polyethylene, polypropylene, fluorinated ethylene propylene, polyester, polysulfone, and the like. Referring to FIGS. 2A, 2B, and 3B, the cam lock 54 preferably rotates smoothly and with minimum friction within the hub 56. The cam lock 54 further comprises features on its proximal surface which engage the hub 56 of FIG. 2A to restrain the cam lock 54 to rotate concentrically within the hub 56. The hub 56 further comprises a slot to permit limited rotational or circumferential motion of the handle 76 where it projects radially through the hub 56. The hub 56 may further comprise optional ratcheting or detents (not shown) to provide for tactile feedback of the extent of cam lock 54 travel. Further, calibration marks can be applied to the hub 56 to allow the user to visualize the extent of travel of the cam lock 54 and infer the amount of radial dilation of the anchors 56. The cam lock 54 is suitable for use with any sheath that requires a radially dilating attachment point to the hub 56. For example, the cam lock 54 is well suited to work with the sheath 10 of FIGS. 1A and 1B.

In yet another embodiment, the sheath 50 is configured for one-handed operation. A trigger assembly or a lever assembly may be attached to the hub 56, which advances the rail slide 62 distally toward the hub 56. The trigger may be squeezed in the hand and released multiple times to permit a ratcheting type advancement of the slide 62 and the ultimate locking attachment of the seal hub 44 to the hub 56.

Referring to FIGS. 1A and 1B the seal hub 24 comprises a housing that is fabricated from the same or similar materials as those used to fabricate the hub 18. These same materials are also used in fabrication of the seal hub 44 in FIGS. 2A and 2B. The seal hub 24 further comprises an elastomeric membrane 42 that is suspended within the seal hub 24. The elastomeric membrane 42 is generally configured as a washer with a central orifice, hole, pinhole, slits, or an opening, capable of accepting instruments therethrough and sealing to the outer surface of said instruments. The central orifice of the elastomeric membrane 42 enlarges or shrinks as necessary to accommodate a wide range of instruments. The orifice diameter of the elastomeric membrane, in the unstretched state ranges from 0.05 inches to 1 inch. The elastomeric membrane is fabricated from materials such as, but not limited to, silicone elastomer, thermoplastic elastomer, polyurethane, latex rubber, and the like. The materials are preferably very soft, elastomeric, and lubricious. The elastomeric membrane is preferably coated with a lubricant such as silicone oil or PTFE to minimize friction on passage of an instrument.

Referring to FIGS. 1B and 2B, in an embodiment of the sheath 10 or 50 the mesh 12 is sheathed with an elastomeric seal layer (not shown) fabricated from materials such as, but not limited to, silicone elastomer, C-Flex thermoplastic elastomer, polyurethane, or the like. The elastomeric seal layer fully encloses and seals the mesh tube 12 which otherwise has a plurality of channels of communication between the inner lumen and the exterior aspect of the mesh tube 12. The elastomeric seal layer may be disposed either on the inner diameter or the outer diameter of the mesh tube 12. In another embodiment, the seal layer is substantially inelastic but is folded in the radially compressed configuration and unfolded in the radially expanded configuration. Such unfurling seal layer is similar in structure to an angioplasty balloon and is coupled either to the inner diameter or outer diameter of the mesh tube 12. The unfurling seal layer is fabricated from materials such as, but not limited to, PTFE, polyurethane, polyethylene, polypropylene, polyamide, polyamide, polyester, and the like. The wall thickness of the unfurling seal layer may range from 0.0002 to 0.010 inches and is preferably between 0.0005 and 0.005 inches. The seal layer may also be created by dipping the mesh 12 within a polymeric material that fills the spaces or interstices of the mesh 12 with sealing elastomer.

Referring to FIGS. 2A, 1A and 2B, provision for use of the sheath 50 over a guidewire is provided either by inserting the sheath 50 over the guidewire. In this embodiment, the guidewire (not shown) is inserted through the guidewire lumen 40 of the obturator 26.

FIG. 4A illustrates a side view of another embodiment of a sheath 100. The sheath 100 comprises a mesh 102, a plurality of standoffs 104, a plurality of pull wires 108, a hub 106, a winding spool 110, a plurality of anchors 112, an obturator 114, and a plurality of pulleys 116. In this embodiment, pull wires 108 are wound onto a spool 110. The spool 110 is coupled to the hub 106 in such a way that it is constrained to rotate circumferentially but not translate along the longitudinal axis of the hub 106. The anchors 112 are constrained to move radially inward or outward within the hub 106 and are coupled at their radially innermost aspect to the standoffs 104. The pull wires 108 are routed from the spool 110 through the hub 106 and then route proximally through pulleys 116 on the proximal end of the mesh 102. The pull wires 108 then route longitudinally to attachment points 118 at the distal end of the mesh 102.

FIG. 4B illustrates the sheath 100 of FIG. 4A wherein the mesh 102 has been radially dilated and axially compressed. When the spool 110 is rotated about the hub 106, the pull wires 108 are wound up on the spool 110 with a resulting tension between the distal end of the sheath 100 and the proximal end of the sheath 100. The proximal end of the mesh 102 moves toward the distal end to shorten the sheath 100 axially and expand the working length radially. The standoffs 104, which are coupled to the anchors 112, which cannot move longitudinally relative to the hub 106, prevent the distal end of the mesh 102 from moving relative to the hub 106. Because of this fixation configuration, the proximal end preferentially moves toward the distal end of the mesh 102. Ratcheting mechanisms, friction mechanisms, or lock mechanisms coupled to the hub 106 and interacting with the spool 110 provide for maintenance of spool 110 location at the desired position relative to the hub 106. The pull wires 108 are fabricated from materials such as, but not limited to, Kevlar, polyamide, polyester, polyurethane, polyethylene, polypropylene, and the like. The pull wires 108 may further be braided or have a monofilament structure. The pull wires 108 may be coated with polytetrafluoroethylene, silicone oil, or the like to reduce friction as they slide through holes or slots in the hub 106.

FIG. 4C illustrates a side view of the obturator 114. The obturator 114 comprises a shaft 120, a nose cone 122, a plurality of hooks, barbs, or prongs 124, a handle 128, a control rod 130, and a button 126. The obturator 114 is an axially elongate structure with a proximal end and a distal end. The obturator 114 preferably further comprises a guidewire lumen (not shown) which is a through lumen extending through the center of the obturator 114 and which can pass, with little or no friction, standard medical guidewires up to and including those of 0.038 inches diameter and larger. The button 126 is coupled to the control rod 130 which may be manually actuated relative to the handle 128 by the user. The control rod 130 is operably connected to the barbs 124 and advancement of the control rod 130 causes the barbs 124 to extend radially outward to grab the mesh of a sheath. Retraction of the control rod 130 causes retraction of the barbs into the shaft 120. In another embodiment, the obturator 114 can be configured to operate in reverse such that advancement of the control rod 130 causes retraction of the barbs 124. In other embodiments, the button 126 may be a lever or other actuator, rather than a pushbutton. The obturator 114 may either be flexible or rigid, depending on the application. The shaft 120 of the obturator 114 preferably has column strength even if it is flexible. The shaft 120 may be made even more flexible by fabricating it with an exterior set of circumferential grooves or spirals or it may be braided. A flexible shaft 120 generally benefits from a flexible control rod 130 that also has column strength.

The obturator shaft 120 and control rod 130 in this embodiment is fabricated from materials such as, but not limited to, PEBAX, polyethylene, polypropylene, or polyamide, preferably further comprising a braided or coiled reinforcement of materials such as, but not limited to, stainless steel, titanium, nitinol, or the like. The shaft 120 may be made substantially flexible by fabricating it from polymeric materials and not from metals. The control rod 130 may further be fabricated from metals such as stainless steel, cobalt nickel alloys, titanium, and the like.

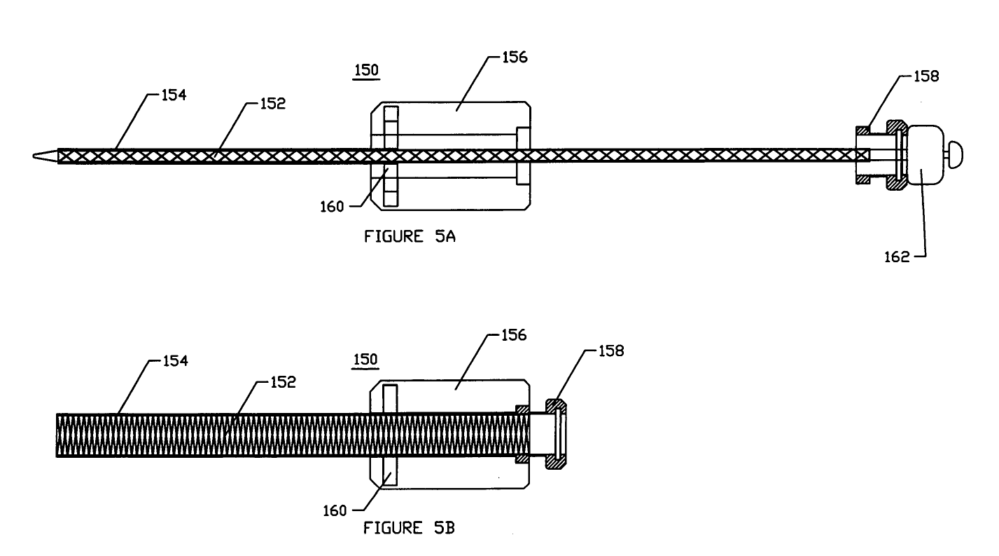

FIG. 5A illustrates another embodiment of a sheath 150, comprising a mesh 152, a plurality of standoffs 154, a hub 156, a seal hub 158, a plurality of radial anchors 160 and an obturator 162 in its radially collapsed configuration.

The mesh 152 can be fabricated from shape memory materials such as, but not limited to nickel titanium alloy, or nitinol. The composition of the nitinol may be either equiatomic or nickel-rich. Wires of nitinol are braided or formed in a counterwound spiral to form an axially elongate shape. The wires may be either round in cross-section or they may be rectangular in cross-section. The nitinol is preferably heat treated and formulated to generate austenitic characteristics above a temperature known as the Austenite finish temperature, or Af. Above the Af temperature, the wires of the mesh will seek to return to a pre-determined shape. Below Af, the material is not austenitic but is in transition. Below a temperature known as the Austenite start temperature (As), the material has malleable characteristics and is described as being Martensitic. In one embodiment, the wires are disposed to run in a generally longitudinal direction when the mesh 152 is in its radially collapsed configuration. In such an embodiment, the wires do have some circumferential directionality but a side view of the mesh 152 would preferably show the wires as subtending an angle of approximately 45 degrees or less relative to the longitudinal axis of the mesh 152.

FIG. 5B illustrates the sheath 150 of FIG. 5A wherein the mesh 152 has been heated to a temperature above Af. Heating may be accomplished by exposing the mesh 152 to body temperature if the Af temperature is set somewhat lower, say in the range of 20 to 32 degrees centigrade. Since the working length of the sheath 150 is inserted into the body, it heats to above austenite finish and becomes a very strong spring biasing itself to its pre-determined shape. The heating may also be accomplished using Ohmic or resistive electric heating by passing current through nickel-chrome wire or even through the nitinol. Maximum practical temperatures for Ohmic heating are in the range of 35° C. to 42°C. The heating can be removed and the mesh 152 will maintain its superelastic or pseudoelastic nature by virtue of hysteresis. Hysteresis is a property of nitinol that allows the material to retain its austenitic characteristics at lower temperatures during the cooling part of the cycle than are obtainable during the heating part of the cycle. The mesh 152 has formed into a more circumferentially oriented and axially compressed configuration than the mesh of FIG. 5A. In the radially expanded configuration, the filaments or wires of the mesh subtend angles generally greater than 45 degrees relative to the longitudinal axis of the mesh 152. The length of the sheath 150 has shortened but, because of the standoffs 154 and the anchors 160, the shortening occurs at the proximal end of the sheath 150. The seal hub 158 has moved distally and has been locked into the hub 156 to maintain the radially dilated configuration.

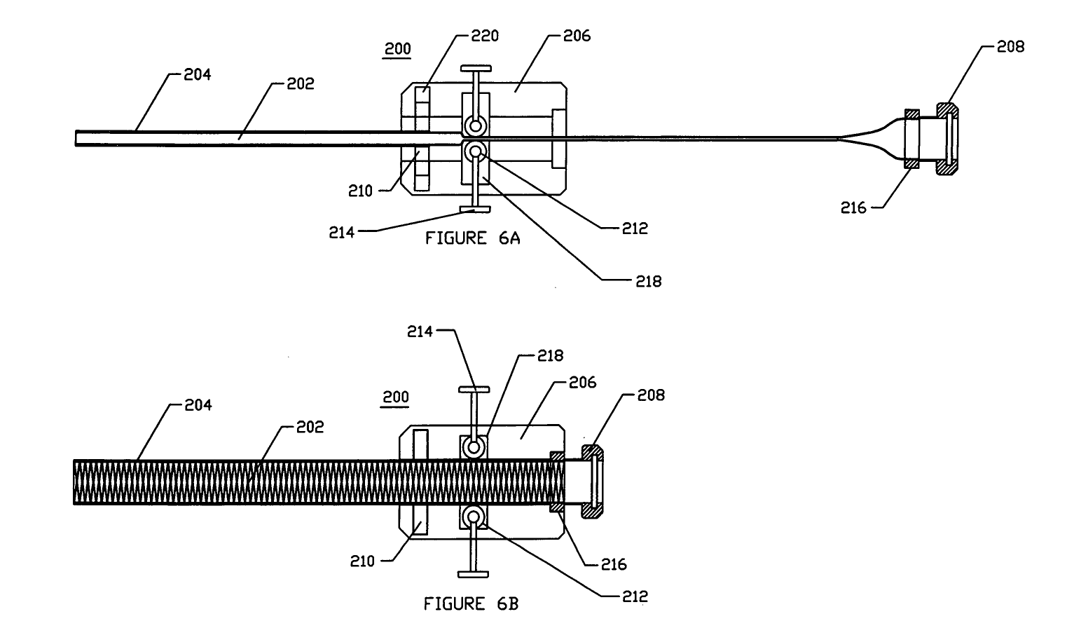

FIG. 6A illustrates an embodiment of a sheath 200 wherein the mesh 202 is routed through a pair of pinch rollers 212 located in the hub 206. The sheath 200 further comprises a plurality of anchors 210, a plurality of standoffs 204, and a seal hub 208. The anchors 210 slide within slots or channels 220 within the hub 206. The pinch rollers 212 are biased, using either springs or locking mechanisms on the pinch roller control mechanism 214, to compress around the mesh 202, which is flattened where it passes through the pinch rollers 212. The pinch rollers 212 move radially inward and outward within pinch roller slots 218 within the hub 206. The excess mesh material 202 between the pinch rollers 212 and the seal hub 208 extends out the back of the hub 206. In an embodiment, the excess mesh material 202 extends out the proximal end of the hub 206 in a linear fashion. In another embodiment, the mesh 202 may be rolled onto a cassette (not shown) that is coupled to the hub 206 to minimize the bulk and extent of the sheath 200. In the embodiment where the mesh 202 is contained within a cassette, the seal hub 208 is preferably rotationally movable about the axis of a spool within the cassette so that the orientation of the seal hub 208 may accommodate that of the proximal free end of the mesh 202. A bayonet mount 216 or other type of releasable lock is comprised by the seal hub 208 and mating regions of the hub 206. The seal hub 208, when attached to the hub 206, is preferably sealed against fluid leakage at the lock interface.

FIG. 6B illustrates the sheath 200 of FIG. 6A wherein the mesh 202 has been advanced distally by the pinch rollers 212 disposed within slots 218 comprised by the hub 206. The seal hub 208 has been locked to the hub 206 by way of a bayonet mount 216. The pinch rollers 212 have been unlocked by the control mechanism 214 and have moved radially outward within the slots 218 to permit the mesh 202 to fully expand. The anchors 210 which are coupled to the standoffs 204 have moved radially outward to permit the mesh 202 to fully expand. The seal hub 208 has sealed to the hub 206 in a fluid-tight manner and is capable of accepting instrumentation through its central lumen. In this embodiment, the filaments of the mesh 202 are disposed substantially in the circumferential direction but with some longitudinal disposition. Accordingly, the adjacent filaments within the mesh 202 are tightly packed and present a sheath 200 with maximum hoop strength and resistance to radial compression.

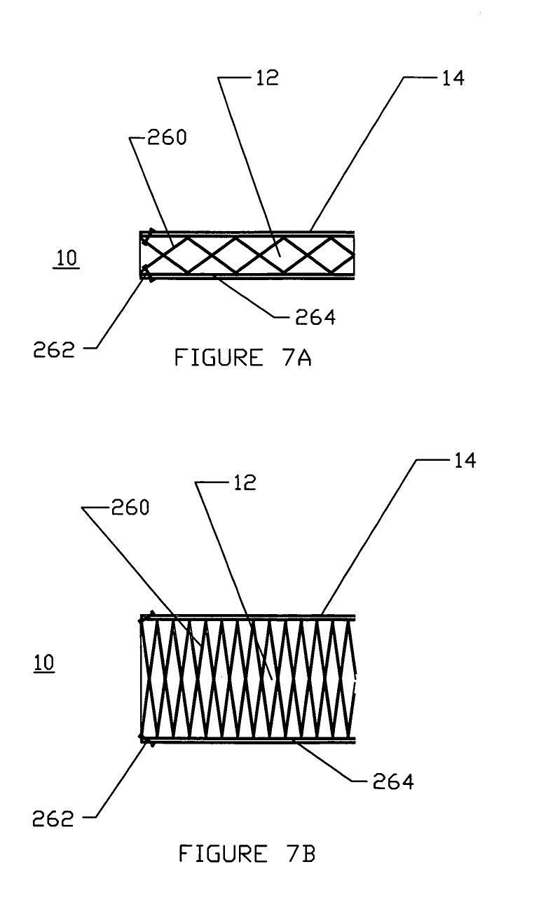

FIG. 7A illustrates an embodiment of the mesh 12 of the sheath 10 shown in FIG. 1A. The mesh 12 has been longitudinally elongated or stretched and so its diameter or radius is minimized. The wires or filaments 260 are disposed to run in a generally longitudinal direction when the mesh 12 is in its radially collapsed configuration. In this embodiment, the wires do have some circumferential directionality but a side view of the mesh 12 would generally show the wires as subtending an angle of approximately 45 degrees or less relative to the longitudinal axis of the mesh 12. The wires comprising the mesh 12 may have a round cross-section or they may have a rectangular or other cross-sectional configuration. The standoffs 14 are shown along with a plurality of distal fixation mechanisms 262, which affix the standoff generally to the distal end of the mesh 12. The mesh 12 is further covered by a containment layer or barrier 264. In this preferred embodiment, the containment layer 264 is disposed internal to the standoff 14. In another embodiment, the containment layer 264 is located external to the standoff 14.

FIG. 7B illustrates the mesh 12 of FIG. 7A wherein the mesh 12 has been compressed along its longitudinal axis causing the diameter or radius of the mesh 12 to increase. The individual filaments 260 of the mesh 12 have formed into a more circumferentially oriented and axially compressed configuration than the mesh of FIG. 7A. In the radially expanded configuration, the filaments or wires 260 of the mesh subtend angles generally greater than 45 degrees relative to the longitudinal axis of the mesh 12. The filaments or wires 260 are closely packed next to each other along the longitudinal axis of the sheath 10, in this configuration. The containment layer 264 has stretched or unfurled to accommodate the diameter change in the mesh 12. The standoffs 14 have expanded diametrically to adjust to the new mesh 12 diameter. The fixation mechanisms 262, two of which are shown, have rotated slightly to adjust to the new mesh 12 configuration. The fixation mechanisms 262 may be sutures or wires routed through holes or slots in the standoffs 14 and around individual filaments 260. The fixation mechanisms 260 may further be rivets, “U” bolts, or other types of devices suitable for fixing fabrics to a rigid structure. In another embodiment, the fixation mechanism 260 is a weld or adhesive structure such as that formed by ultraviolet light curable adhesives which are manufactured by companies such as Loctite or Dymax and which often are based on polyurethane.

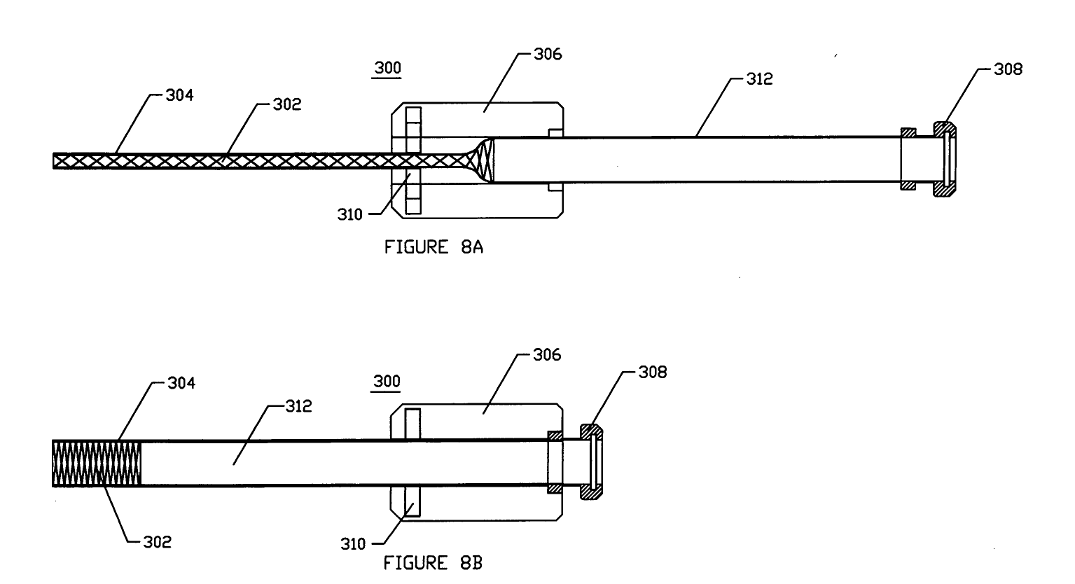

FIG. 8A illustrates another embodiment of the sheath 300, comprising a rigid pusher tube 312, at least one standoff 304, at least one radially movable anchor 310, a hub 306, a seal hub 308, and a mesh 302, wherein the mesh 302 projects out the distal end of the hub 306. The proximal end of the mesh 302 is coupled to the rigid tube 312 by welding, insert molding, bonding with adhesives, fasteners, or the like. The rigid tube 312 has substantially the same internal diameter as that of the fully radially expanded mesh 302. It is beneficial to have the mesh 302 extend somewhat into the hub 306 to allow for a transition in diameter from its radially contracted working length to fully radially expanded within the hub 306. The rigid tube 312 extends proximally from the hub 306 and terminates with an coupled seal hub 308. The seal hub 308 is capable of releasably attaching to the hub 306. The rigid pusher tube 312 slides through the internal bore of the hub 306 on bushings or bearings (not shown) that are configured to accept the rigid tube 312 so that it moves back and forth axially but does not torque or move out of the axis of the sheath 300. This embodiment can be used with or without an obturator (not shown). The sheath 300, in a preferred embodiment, comprises fingers or slotted leafs (not shown) that project distally from the rigid tube 312 along the inner diameter of the mesh 302 for a distance no greater than that of the length of the compressed mesh 302. The fingers serve the function of acting like a strain relief to coerce the mesh 302 to expand radially as the rigid pusher tube 312 is advanced distally. The fingers slide along the longitudinal axis with the rigid pusher tube 312. The fingers are preferably fabricated from materials such as, but not limited to, spring hardened stainless steel, nitinol, Elgiloy, or the like. The fingers are slotted to permit expansion or contraction radially as required.

FIG. 8B shows the sheath 300 of FIG. 8A in its diametrically expanded state. To expand the working length of the mesh 302, the rigid tube 312 is optionally unlocked from the hub 306 and advanced distally until the seal hub 308 is latched onto the hub 306. The anchors 310 have been unlocked and have either been forcefully moved or have moved passively to their radially dilated configuration. In this radially expanded embodiment, the rigid tube 312 will extend distal to the hub 306 for a substantial distance and only the distal most portion of the working length will be mesh 302. The length of the rigid tube 312 is approximately that of the working length of the sheath 300, that is, the distance from the distal end of the mesh 302 and standoffs 304 to the distal end of the hub 306. This embodiment has the advantage of extremely high hoop strength, far in excess of that that can be generated with a filamentous mesh only. However, the filamentous mesh 302 comprises the entire working length of the sheath 300 during the diametrically collapsed configuration and only a part of the working length during the diametrically expanded configuration. This configuration is especially useful for procedures that require penetration of non-paralyzed muscles or ligaments or even spaces between bony structures. The mesh 302 preferably comprises a seal or containment layer (not shown) and the distal end of the mesh 302 is coupled to standoffs 304 that provide resistance to forces directed distally thereon. The standoffs 304 are radially expandable and longitudinally restrained by anchors 310 within the hub 306. The standoffs 304 further are preferably movable or lockable between the radially contracted and expanded state.

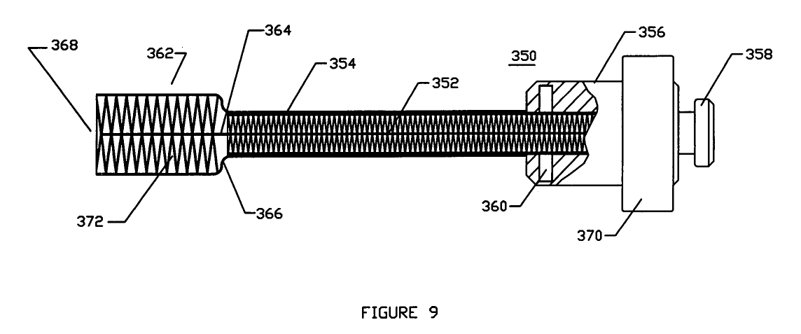

FIG. 9 illustrates another embodiment of an expandable sheath 350 further comprising a dome instrument 362 expanded at the distal end of the sheath 350. In one embodiment, the dome instrument 362 is configured to surround an organ or tissue and permit therapy or removal of that tissue or organ. The dome instrument 362 comprises a tubular mesh 372, which is expanded radially by axial compression. The tubular mesh 372 of the dome instrument 362 expands to a diameter larger than that of the mesh 352 used for the main body of the sheath 350. In a preferred embodiment, the tubular mesh 372 of the dome instrument collapses to a diameter similar to that of the tubular mesh 352 used on the sheath 350 working length. In other embodiments, the dome instrument 362 may be expanded using devices such as, but not limited to, balloon structures, unfurling thin wall sheaths with a cam or control rod actuator, or the like. The dome instrument 362 further comprises a plurality of pull wires 364, a fluid impermeable containment layer 366, and a dome lumen 368. The pull wires 364 are activated at the region of the hub 356 of the sheath 350. A winding spool 370 provides a control mechanism to put the pull wires 364 in tension and compress the distal end of the dome instrument 362 toward the proximal end of the dome instrument 362. The dome instrument 362 in one embodiment may foreshorten or it may be restrained from foreshortening when it is radially dilated by use of standoffs 354 which are attached to anchors 360. Using this technique and apparatus, a dome instrument 362 whose diameter is larger than that of the sheath 350 may be created at the distal end of the sheath 350. The sheath 350 further comprises a seal hub 358.

Referring to FIG. 9, the containment layer 366 is preferably an elastic or unfurling inelastic membrane that completely surrounds the dome instrument 362 and which seals to the sheath 350 to prevent the escape of fluids or contaminants from the dome lumen or the sheath 350 central lumen. The containment layer 366 expands diametrically with the dome instrument 362 wall and contracts, actively if it is elastomeric, or passively if it is furled, when the dome instrument 362 wall is contracted radially. In another embodiment, the distal edge of the dome instrument 362 is configured to be atraumatic to tissues against which it may be pressed. The atraumatic edge of the dome instrument 362 is preferably created by folding the containment layer 366 over the distal edge of the dome instrument 362 or by inverting the dome instrument 362 inside itself. The atraumatic edge may also be created by the addition of a bead of soft material such as silicone elastomer or polyurethane. The distal atraumatic edge of the dome instrument 362 may also be configured by putting an edge radius, a fold, a separate distal edge piece bonded thereto using heat or adhesives, or a bend in the material of the wall of the dome instrument 362 at its distal edge.

Another embodiment of the invention is a method of use where the sheath is inserted into an animal, mammal, or preferably a human. A small incision is first made into the skin and the tissue is dissected or tunneled, either bluntly or sharply, to the target site where surgical repair or diagnosis is required. The sheath is inserted with or without the aid of a guidewire having been first inserted into the tunnel, to the target site. The working length of the sheath, that is the length of the sheath that projects distal to the hub, is expanded diametrically by compressing the proximal end of a mesh comprised within the sheath toward the hub or toward the distal end of the sheath. The proximal end of the mesh is secured to a seal hub that is preferably reversibly attached to the hub of the sheath to lock the axially compressed and radially dilated configuration in place. Instruments can be passed through the seal hub and the central lumen of the mesh toward the target site to provide therapeutic or diagnostic procedures while seals within the seal hub prevent the escape of fluids through these structures. The procedure can be reversed to remove the sheath following reduction in diameter of the working length of the sheath. The compression of the mesh toward the hub or toward the distal end of the sheath can be done with two hands or with one hand by using controlling devices such as triggers or electric motors. In a preferred method, the distal end of the mesh is coupled to the hub by one or more standoffs that are themselves coupled to radially sliding anchors within the hub. The standoffs may also be coupled to the hub by rocker arms or crankshafts or leaf spring arrangements which may provide for mechanically simpler radially movable but longitudinally fixed attachments. The mesh is preferably not coupled to the hub in its central region, that is the region of the mesh substantially between the proximal end and the distal end. By adhering to these principles, a braided surgical retractor can be made to expand diametrically without a substantial change in working length.

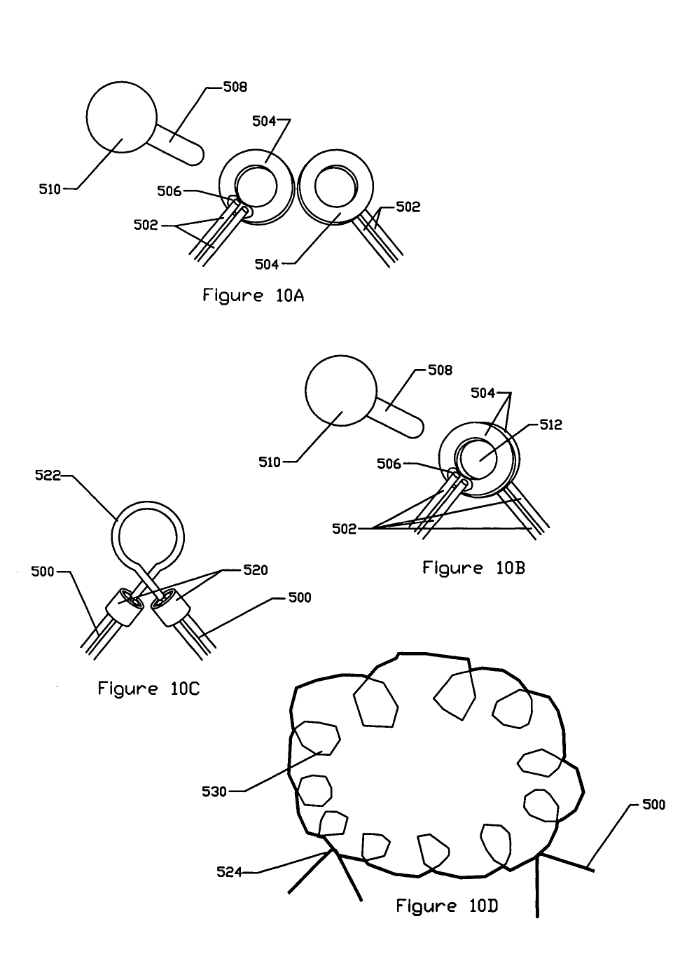

FIG. 10A illustrates a close-up view of an embodiment of two of the ends 500 of the mesh 12 of the sheath 10 (for example, as shown in FIGS. 1A and 1B). In this embodiment, the ends 500 comprise two filaments 502 each and are tipped by washers 504, which are coupled to the filaments 502 by welds 506. The ends 500 can comprise between 1 and 20 of the filaments 502. A pin 508 is shown prior to being inserted through the washers 504 to constrain the washers 504 in a given translational position. The pin 508 further comprises an expanded end 510 to maintain the pin 508 axially constrained within the washers 504. The ends 500 are free to rotate in this embodiment to the extent permitted by the filaments 502 and the pin 508 and washer 504 assembly. The attachment of the washer 504 to the ends 500 is generally performed using a plasma weld, laser weld, adhesive weld, mechanical attachment, crimp attachment, or other suitable joining mechanism.

FIG. 10B illustrates a close-up view of the two ends 500 of FIG. 10A wherein the two washers 504 have been brought into coaxial alignment, one on top of the other, so that the pin 508 can be inserted through the central hole 512 of the washers 504. The leading edge of the pin 508, that end that is inserted through the washers 504, does not have the expanded or flared configuration 510. Following insertion of the pin 508 through the washers 504, the leading end is then flared 510 or a nut or other flare is mechanically attached. The pin 508 with its expanded or flared ends 510 functions like a rivet or axle.

FIG. 10C illustrates a close-up view of two ends 500 which have been interconnected by a flexible loop or coil 522. In the illustrated embodiment, the loop or coil 522 is coupled to the ends 500 by a crimp collar 520. The loop or coil 522 can be a spring material such as spring hardened stainless steel, cobalt-nickel-chrome alloy, nitinol, or the like, or it can be a malleable strand fabricated from annealed stainless steel, polymer, titanium, or the like.

FIG. 10D illustrates a close-up view of two ends 500, which are interconnected by a simple weld or bond 524. The ends are then all joined by a coil spring 530. This embodiment provides a ring end structure to the sheath 10. The coil spring 530, in another embodiment, can also be an elastic band or collar or an unfurling inelastic band or collar. A key advantage of the ring or band end structure is to blunt any sharp edges that might be present with the ends 500.

The present invention may be embodied in other specific forms without departing from its spirit or essential characteristics. For example, the sheath may include instruments coupled integrally to the interior central lumen of the mesh, rather than being separately inserted, for performing therapeutic or diagnostic functions. The hub may comprise tie downs or configuration changes to permit attachment the hub to the skin of the patient. The sheath can be used for endovascular or endoluminal access and can comprise appropriate hemostatic seals and valves coupled to the proximal end. The described embodiments are to be considered in all respects only as illustrative and not restrictive. The scope of the invention is therefore indicated by the appended claims rather than the foregoing description. All changes that come within the meaning and range of equivalency of the claims are to be embraced within their scope.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. In addition, while a number of variations of the invention have been shown and described in detail, other modifications, which are within the scope of this invention, will be readily apparent to those of skill in the art based upon this disclosure. It is also contemplated that various combinations or subcombinations of the specific features and aspects of the embodiments may be made and still fall within the scope of the invention. Accordingly, it should be understood that various features and aspects of the disclosed embodiments can be combine with or substituted for one another in order to form varying modes of the disclosed invention. Thus, it is intended that the scope of the present invention herein disclosed should not be limited by the particular disclosed embodiments described above, but should be determined only by a fair reading of the claims that follow.

What is claimed is:

1. An access sheath for expanding an opening in biological tissue from a first cross-sectional area to a second larger cross-sectional area, the sheath comprising:
a hub having a distal end and a proximal end and an opening extending therethrough;
a substantially tubular mesh extending through the hub and comprising a distal portion that extends from the distal end of the hub and a proximal portion that extends proximally from the proximal end of the hub;
a radially expandable standoff that extends distally from the hub and is coupled to a distal portion of the mesh and to the hub; and
a compression mechanism configured to advance the proximal portion of the mesh distally towards the hub causing axial compression and diametrical expansion of the mesh.

2. The sheath of claim 1, further comprising a containment layer that is substantially impermeable to liquids and is coupled to the mesh.

3. The sheath of claim 2, wherein the containment layer is disposed on the exterior of the mesh.

4. The sheath of claim 1, further comprising a seal hub that is coupled to the proximal portion of the mesh.

5. The apparatus of claim 4, further comprising a lock configured to selectively couple the seal hub to the hub.

6. The sheath of claim 4, further comprising a telescoping member extending between the seal hub and the proximal end of the hub.

7. The sheath of claim 4. further comprising a slide member extending between the seal hub and the proximal end of the hub.

8. The sheath of claim 4, wherein the seal hub is configured be disconnected from the hub and withdrawn proximally to allow the distal portion of the mesh to become smaller in diameter.

9. The sheath of claim 1, wherein the compression mechanism comprises a pinch roller mechanism.

10. The apparatus of claim 1, wherein the mesh is configured to be advanced distally by a shape memory transition from a martensitic to an austenitic state.

11. The sheath of claim 1, further comprising an obturator that extends through the mesh.

12. The sheath of claim 1 wherein the mesh and the at least one standoff are configured to bend.

13. A method of providing access to a surgical site; comprising:
inserting a sheath, which comprises a hub and an expandable tubular member that extends through the hub, into an incision;
advancing a distal portion of the tubular member to a target depth; and
expanding the distal portion of the tubular member diametrically while simultaneously advancing a proximal portion of the tubular member distally toward the hub.

14. The method of claim 13, further comprising inserting an obturator into the incision along with the sheath.

15. The method of claim 13, further comprising the step of inserting the sheath and an obturator over a guidewire.

16. The method of claim 13, further comprising inserting at least one instrument through a lumen of the sheath.

17. The method of claim 13, further comprising reducing the diameter of the sheath and thereafter removing the sheath from the patient.

18. A surgical access device for expanding an opening in a patient, comprising:
a hub having a distal end and a proximal end and an opening extending therethrough;
a radially expandable tubular body comprising a distal portion that extends distally of the hub, a proximal portion that extends proximally of the hub, and an axial lumen therethrough; and
means for applying an axially compressive force to the tubular body to reversibly expand the axial lumen from a first, smaller, diameter to a second, larger diameter, without substantially shortening the axial length of the distal portion of the tubular body.

19. A method of providing access to a surgical site; comprising:
providing a sheath having a hub and an expandable tubular member comprising a distal portion and a proximal portion, the distal portion extending distally of the hub and the proximal portion extending proximally of the hub;
inserting the distal portion of the tubular member into an incision;
advancing the distal portion of the tubular member to a target depth; and
expanding the distal portion of the tubular member diametrically without substantially changing the axial length of the distal portion with respect to the hub.

20. The method of claim 19, wherein expanding the distal portion of the tubular member further comprises advancing the proximal portion of the tubular member towards the hub.
